(19) United States
(12) Patent Application Publication
(10) Pub. No.: US 2026/0014388 A1
(43) Pub. Date: Jan. 15, 2026

(54) SPECTRAL ABSORPTION MEASUREMENT DEVICE

(71) Applicant: LUMITEX, INC., Brecksville, OH (US)

(72) Inventor: Michael KERNS, Brecksville, OH (US)

(73) Assignee: LUMITEX, INC., Brecksville, OH (US)

(21) Appl. No.: 18/996,392

(22) PCT Filed: Sep. 12, 2023
(86) PCT No.: PCT/US2023/073922

§ 371 Date: Jan. 17, 2025

Related U.S. Application Data
(60) Provisional application No. 63/375,400, filed on Sep. 13, 2022.

(51) Int. Cl. A61N 5/06

(52) U.S. Cl. CPC A61N 5/0616; A61N 2005/0626; A61N 2005/0659; A61N 2005/0663

(57) ABSTRACT

A phototherapy system and spectral absorption measurement device are provided for adjusting parameters used to generate phototherapeutic light by determining a tissue's spectral absorption characteristics by employing detector assemblies that analyze the tissue's effect on specific wavelengths of light.

### RELATED APPLICATIONS

This application claims the benefit of U.S. 63/375,400 filed on Sep. 13, 2022, which is herein incorporated by reference in its entirety.

### TECHNICAL FIELD

The present disclosure relates generally to phototherapy and more particularly to measuring tissue absorption of electromagnetic radiation.

### BACKGROUND

Phototherapy (also referred to as photobiomodulation) devices apply therapeutic light to a skin surface. When the therapeutic light reaches the skin, the light is (1) reflected and becomes useless for therapy; (2) penetrates the skin but is absorbed by a non-target tissue (e.g., is converted into heat in the non-target tissue); or (3) penetrates the skin and is absorbed by the target tissue (i.e., resulting in treatment).

### SUMMARY

Therapeutic light that is reflected by the skin (referred to as reflective losses) can be actively monitored with light sensors. If the optical power of the emitted therapeutic light is known (referred to as the input power), the difference between the known input power and the measured reflective losses is equal to the combination of outcomes (2) and (3) (i.e., light penetrating the skin and being absorbed by a non-target tissue or a target tissue). With only the measured reflective therapeutic light, it is not possible to know how much of the emitted therapeutic light is absorbed by the target tissue.

Knowing the makeup of the tissue being irradiated and comparing the tissue makeup to a predetermined (e.g., an experimentally derived) map of tissue optical parameters can be used to determine the proportion of therapeutic light absorbed by the target tissue, giving a breakdown of where optical energy is absorbed, which can be used as feedback to control phototherapy.

The present disclosure provides a spectral absorption measurement device configured to determine a makeup of tissue being illuminated during phototherapy, identify as effective therapeutic light an amount of therapeutic light being absorbed by a target tissue, and modify phototherapy parameters based on the identified effective therapeutic light.

While a number of features are described herein with respect to embodiments of the invention; features described with respect to a given embodiment also may be employed in connection with other embodiments. The following description and the annexed drawings set forth certain illustrative embodiments of the invention. These embodiments are indicative, however, of but a few of the various ways in which the principles of the invention may be employed. Other objects, advantages and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention in which similar reference numerals are used to indicate the same or similar parts in the various views.

FIG. 1 is a schematic diagram of a phototherapy system including a light source and a spectral absorption measurement device.

FIG. 2 is a schematic view of a spectral absorption measurement device including detector assemblies positioned on a tissue.

FIG. 3 is a schematic view of a positioning of a light emitter and light sensors of a detector assembly.

The present invention is described below in detail with reference to the drawings. In the drawings, each element with a reference number is similar to other elements with the same reference number independent of any letter designation following the reference number. In the text, a reference number with a specific letter designation following the reference number refers to the specific element with the number and letter designation and a reference number without a specific letter designation refers to all elements with the same reference number independent of any letter designation following the reference number in the drawings.

### DETAILED DESCRIPTION

The present disclosure provides a phototherapy system and spectral absorption measurement device for adjusting parameters used to generate phototherapeutic light by determining a tissue's spectral absorption characteristics by employing detector assemblies that analyze the tissue's effect on specific wavelengths of light.

According to a general embodiment, a spectral absorption measurement device is provided including multiple detector assemblies for detecting spectral absorption characteristics of tissue. Each detector assembly is keyed to a wavelength of light and includes a light emitter for emitting identifying light of the keyed wavelength and a light sensor for detecting the identifying light returning from the tissue. The spectral absorption measurement device also includes processor circuitry for analyzing the detected returning identifying light to determine the spectral absorption characteristics of the tissue, identify an effect of the tissue on therapeutic light, and modulate phototherapy parameters based on the identified effect on the therapeutic light.

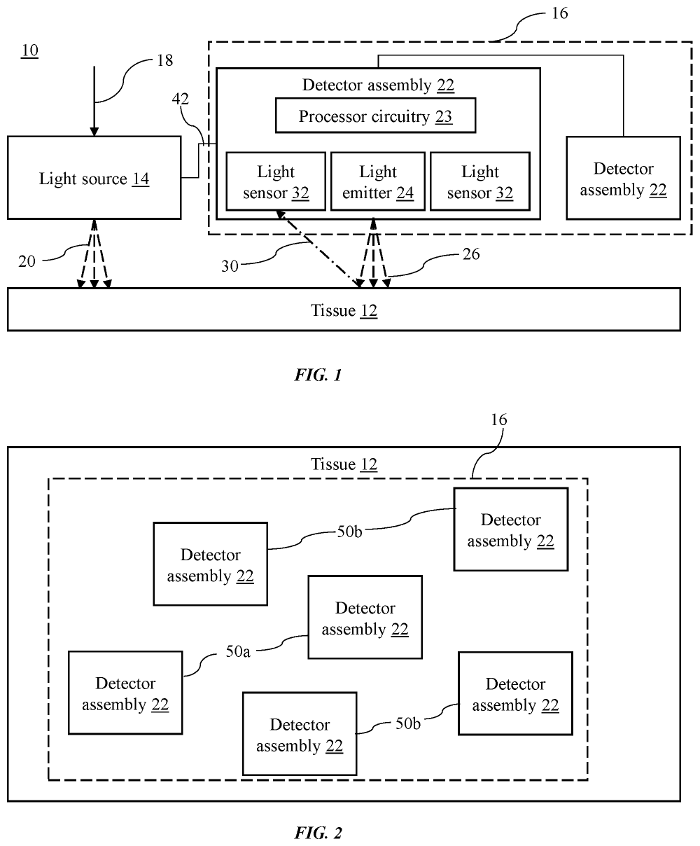

Turning to FIG. 1, a phototherapy system 10 is shown for providing targeted phototherapy to tissue 12. The phototherapy system 10 includes a light source 14 and a spectral absorption measurement device 16. The light source 14 receives phototherapy parameters 18 and emits light 20. The emitted light 20 includes phototherapeutic light having at least one phototherapeutic wavelength based on the received phototherapy parameters 18. The spectral absorption measurement device 16 determines spectral absorption characteristics of the tissue 12 and adjusts the phototherapy parameters 18 based on the determined spectral absorption characteristics.

The spectral absorption measurement device 16 includes multiple detector assemblies 22 and processor circuitry 23. Each assembly 22 is keyed to a particular wavelength of light. These assemblies 22 include a light emitter 24 for emitting identifying light 26 that includes the keyed wavelength of light. When this identifying light 26 interacts with the tissue 12, a portion 30 of the identifying light returns from the tissue 12. This returning light 30 is then detected by at least one light sensor 32 within the assembly that is sensitive to its respective keyed wavelength.

The processor circuitry 23 of the spectral absorption measurement device 16 determines an input property of the emitted identifying light 26 (e.g., offering insights into the primary characteristics of the light as it interacts with the tissue). The processor circuitry 23 also determines an output property of the detected returning light 30 (e.g., presenting a clearer understanding of how the light has been influenced by interaction with the tissue 12). Leveraging the determined input and output property of the light, the processor circuitry 23 determines optical tissue properties based on the effect of the tissue on the keyed wavelengths emanating from the detector assemblies. For example, the optical tissue properties may include a ratio of the output property to the input property (e.g., input power to measured output power) for different wavelength.

Based on the optical tissue properties, the processor circuitry 23 determines as spectral absorption characteristics an effect of the tissue 12 on the phototherapeutic wavelength. For example, the spectral absorption characteristics may include a predicted absorption (e.g., as a percentage of the input light) by the tissue of one or more wavelengths of light not included in the identifying light 26 (e.g., the phototherapeutic wavelength). As a culmination of these processes, the circuitry 23 outputs a light effect signal 42 describing modifications to the phototherapy parameters based on the determined spectral absorption characteristics 40.

The light effect signal 42 is received by the light source 14 and the light source 14 modulates the phototherapy parameters based on the light effect signal 42. For example, the light effect signal 42 may specify updated phototherapy parameters that are used by the light source 14. Alternatively, the light effect signal 42 may specify quantitative alterations to the phototherapy parameters. For example, the light effect signal 42 may specify to increase output of one of the phototherapeutic wavelengths by a percentage (e.g., due to reduced absorption by the tissue 12). In another embodiment, the light effect signal 42 may specify an effect of the tissue 12 on different wavelengths of light, which the light source 14 may interpret to modulate the phototherapy parameters. For example, if the phototherapy parameters result in an output of green light and the light effect signal 42 indicates that 20% of green light is being reflected by the tissue 12, then the light source 14 may modulate the phototherapy parameters, such that an amount of green light emitted by the light source 14 is increased by 20%.

The above-described keyed wavelengths may be used by the spectral absorption measurement device to determine the effect of the tissue on the phototherapeutic light. The keyed wavelengths may encompass both phototherapeutic and non-phototherapeutic frequencies. While some configurations ensure that each phototherapeutic wavelength is represented among the keyed wavelengths, allowing for direct interaction measurements, others incorporate probing wavelengths. These probing wavelengths, distinct from the phototherapeutic light, aid in determining the optical properties of the tissue.

In some embodiments, not all phototherapeutic wavelengths are represented among the keyed wavelengths. To illustrate, consider a configuration with five potential phototherapeutic wavelengths. If only four of these are keyed, the fifth is not directly monitored by the detector assemblies. Consequently, the system, referred to as system 10, the effects of tissue on the unkeyed phototherapeutic wavelength is not directly measured. To address this limitation, the spectral absorption measurement device may use the determined effects of the tissue on the keyed wavelengths as a reference. By analyzing these interactions, the processor circuitry may extrapolate the likely effects of the tissue on the unkeyed phototherapeutic wavelength. This allows for the approximation of tissue interactions even when direct measurements are unavailable.

In one embodiment, each of the detector assemblies may accommodate multiple keyed wavelengths of light. For example, the light sensor of each assembly may be configured to detect multiple keyed wavelengths associated with its respective assembly. This multiplexed detection mechanism may allow for serial or simultaneous monitoring of different interactions of the light with the tissue. In this example, the light emitter in each detector assembly may be adjusted to produce identifying light that includes all of the multiple keyed wavelengths specific to its assembly. This means each emitter may cover a broader spectrum of light frequencies during operation, aligning with the expanded sensitivity range of the light sensor.

The light sensors in the phototherapy system may be configured to capture data on the interactions between the keyed light and the tissue. The light sensors may be specifically configured to measure reflectance and backscatter of the keyed light, post tissue interaction. Reflectance measures the fraction of incident light that is reflected off the tissue surface. This information may indicate the tissue's surface properties and the tissue's surface level effect on specific wavelengths of light. Conversely, backscatter may capture the light that is redirected back to the sensor after interacting with the tissue, but not necessarily from the immediate surface. This light may have penetrated deeper and have been scattered multiple times within the tissue before returning. The measurement of backscatter may provide insights into the internal structure and properties of the tissue.

The light sensors may be any suitable device for measuring a property of light, such as backscatter and reflectance. The light sensors may also include filtering devices (e.g., optical filters) for modifying the spectrum of light received by the sensor. The light sensor may detect a wavelength, intensity, power, or any suitable property of light. In one embodiment, each light sensor detects an amount of light for different wavelength ranges. In this embodiment, the sensor data output by the light sensor specifies an amount of light detected for the keyed wavelength of the light sensor. For example, the light sensor may include one or more of photodiodes, phototransistors, Charge-Coupled Devices (CCDs), Photomultiplier Tubes (PMTs), etc. The spectral absorption measurement device may include a single or diverse array of light sensors.

The light emitters may be any suitable device for emitting light. For example, the light emitters may include one or more light emitting diodes (LEDs), organic LEDs (OLEDs), microLEDs, laser diodes, mini-LED, quantum dot (QD)-conversion, phosphor conversion, excimer lamps, multi-photon combination, or Spatial Light Modulator (SLM) wavefront manipulation.

The processor circuitry 23 may process the output property of detected returning light from multiple detector assemblies. For example, the processor circuitry may identify from the keyed wavelengths a reduced wavelength of light representing a wavelength of light that is more preferentially absorbed by the tissue. Furthermore, based on the identified reduced wavelength of light, the processor circuitry may determine specific physiological properties of the tissue. For example, if the identified reduced wavelength of light falls within the infrared (IR) spectrum, the processor circuitry 23 may determine that the tissue has a high water content. Similarly, if the reduced wavelength aligns with the green spectrum of light, the processor circuitry 23 may determine that the tissue includes increased blood content. Conversely, a reduced wavelength in the blue spectrum may indicate heightened melanin levels in the tissue. These determinations, based on specific wavelengths, may allow for a precise understanding of tissue characteristics during phototherapy, which may be used to determine the spectral absorption characteristics of the tissue.

As described above, the input property of the emitted identifying light is used to determine an effect on the light of the tissue. For example, the input property may be compared to the measured output property to directly measure how the property was affected by the tissue. The input property may be determined using any suitable means. For example, the spectral absorption measurement device may include a sensor for directly measuring a property of the light emitted by the light emitter. Similarly, the input property of the emitted identifying light may be determined by the processor circuitry based on driving characteristics of the light source. For example, the processor circuitry may correlate between the current used to drive the light emitter and properties of the light emitted by the light emitter.

In one embodiment, the input property may be an optical power of the emitted identifying light and the output property may be an optical power of the detected returning light. The optical tissue properties may be determined based on the difference between the input property and the output property. In this example, the spectral absorption characteristics may be determined by the processor circuitry comparing the optical tissue properties to predetermined relationships between spectral absorption and tissue parameters to identify an effect of the tissue on the therapeutic light. The effect of the tissue may include a predicted ratio of effective light comprising the phototherapeutic light absorbed by the target tissue and ineffective light comprising the phototherapeutic light not absorbed by the target tissue. The processor circuitry may determine the light effect signal, such that the phototherapy parameters are modulated to ensure that a predetermined amount of the phototherapeutic light is being absorbed by the target tissue. The adjustments to the phototherapy may be compared to a known safe threshold for absorption and therapy may be adjusted accordingly to avoid tissue damage (e.g., subdermal burns).

For example, the processor circuitry may refer to predetermined relationships that exist between spectral absorption and various tissue parameters. These relationships may be stored (e.g., in look up tables) in memory associated with the processor circuitry and may provide a basis for understanding how different types of tissue interact with specific wavelengths of light.

In this example, after the processor circuitry determines the spectral absorption characteristics, the processor circuitry may proceed to a comparison phase. In this phase, the determined characteristics may be cross-referenced with predetermined maps. These maps (e.g., databases, lookup tables, etc.) may contain established relationships between spectral absorption characteristics and different tissue parameters. Examples of tissue parameters could include moisture content, cellular structure, blood flow, and pigmentation among others. By matching the calculated characteristics with known parameters in these maps, the processor circuitry may identify the effect of the tissue on the therapeutic light.

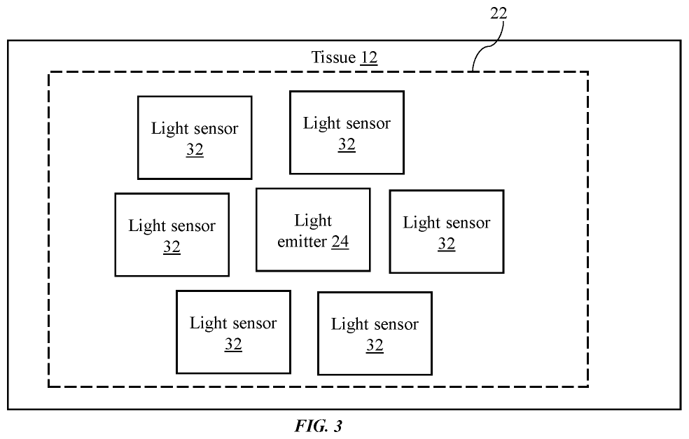

Turning to FIG. 2, the detector assemblies may be classified into groups 50 (e.g., at least two different groups 50a, 50b). Within the same group, each of the detector assemblies may include in the emitted identifying light the same keyed wavelength of light. For example, each light emitter in the group may emit light including a same keyed wavelength of light. Also, within the same group, each of the light sensors may be sensitive to the same keyed wavelength of light. That is, within a group, the light emitters emit the same wavelength of identifying light and the light detectors detect (i.e., are sensitive to) the same wavelength of light.

The different groups of detector assemblies may take any physical relationship relative to one another. For example, the detector assemblies within a group may be spatially intermixed (e.g., to optimize coverage of the tissue being treated by phototherapy) or spatially separated.

The number of groups of detector assemblies may depend on the number of wavelengths desired to characterize the tissue being treated. For example, there may be three or four groups of detector assemblies. For example, the groups of detector assemblies may be keyed to wavelengths of light known to be absorbed by specific tissue components (e.g., water, melanin, fat, hemoglobin, etc.).

Turning to FIG. 3, each of the detector assemblies may include at least three light sensors. The at least three light sensors may have a prescribed relationship to the light emitter. That is, the positioning and configuration of these light sensors may be configured to have a certain relationship with the light emitter. For example, the light sensors may be arranged in such a manner that they form a ring or circle around the central light emitter in the respective detector assembly. Such an arrangement may improve the efficiency of light capture of the emitted light by the light sensors.

All ranges and ratio limits disclosed in the specification and claims may be combined in any manner. Unless specifically stated otherwise, references to “a,” “an,” and/or “the” may include one or more than one, and that reference to an item in the singular may also include the item in the plural.

Although the invention has been shown and described with respect to a certain embodiment or embodiments, equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A phototherapy system for providing targeted light based treatment to tissue comprising:
a light source configured to receive phototherapy parameters and to emit light including phototherapeutic light having at least one phototherapeutic wavelength based on the received phototherapy parameters; and
a spectral absorption measurement device for determining spectral absorption characteristics of the tissue and for adjusting the phototherapy parameters based on the determined spectral absorption characteristics, the spectral absorption measurement device comprising:
detector assemblies, wherein each of the detector assemblies is keyed to a wavelength of light and includes:
a light emitter configured to emit identifying light including the keyed wavelength of light, such that the identifying light interacts with the tissue; and
at least one light sensor sensitive to the keyed wavelength of light and configured to be positioned relative to the tissue, such that returning light is detected by the at least one light sensor, wherein:
the returning light comprises a portion of the identifying light interacting with the tissue that is returned from the tissue; and
processor circuitry configured to:
determine an input property of the emitted identifying light, wherein the input property is an optical power of the emitted identifying light;
determine an output property of the detected returning light, wherein the output property is an optical power of the detected returning light;
determine as optical tissue properties an effect of the tissue on the keyed wavelengths of each of the detector assemblies, wherein the optical tissue properties is determined based on the difference between the input property and the output property;
based on the determined optical tissue properties, determine as the spectral absorption characteristics an effect of the tissue on the at least one phototherapeutic wavelength, wherein:
the spectral absorption characteristics are determined by the processor circuitry comparing the optical tissue properties to predetermined relationships between spectral absorption and tissue parameters to identify an effect of the tissue on the therapeutic light; and
the effect of the tissue includes a predicted ratio of effective light comprising the phototherapeutic light absorbed by the target tissue and ineffective light comprising the phototherapeutic light not absorbed by the target tissue; and
output a light effect signal based on the determined spectral absorption characteristics describing modifications to the phototherapy parameters;
wherein the light source is further configured to modulate the phototherapy parameters based on the light effect signal.

2. The phototherapy system of claim 1, wherein the at least one phototherapeutic wavelength includes at least one wavelength in addition to the keyed wavelengths of the detector assemblies.

3. The phototherapy system of claim 1, wherein each of the detector assemblies includes multiple keyed wavelengths of light, such that:
the at least one light sensor of each of the detector assemblies is sensitive to the multiple keyed wavelengths of the detector assembly; and
the identifying light of the light emitter of each of the detector assemblies includes the multiple keyed wavelengths of the detector assembly.

4. The phototherapy system of claim 1, wherein the light sensors are configured to measure reflectance and backscatter of the keyed light from the tissue.

5. The phototherapy system of claim 1, wherein a combination of the keyed wavelengths of the detector assemblies includes:
at least one of the at least one phototherapeutic wavelength; and
at least one probing wavelength not included in the at least one phototherapeutic light and for determining the optical tissue properties.

6. The phototherapy system of claim 1, wherein the processor circuitry is further configured to:
analyze across the detector assemblies the output property of the detected returning light to identify from the keyed wavelengths as a reduced wavelength of light the key wavelength that is more preferentially absorbed by the tissue; and
determine a physiological property of the tissue based the identified reduced wavelength of light.

7. The phototherapy system of claim 6, wherein the processor circuitry is further configured to determine the physiological property of at least one of:
high water content when the reduced wavelength of light is in an infrared (IR) spectrum of light;
increased blood content when the reduced wavelength of light is in a green spectrum of light; or
increased melanin when the reduced wavelength of light is in a blue spectrum of light.

8. The phototherapy system of claim 1, wherein the input property of the emitted identifying light is determined by the processor circuitry based on driving characteristics of the light source.

9. (canceled)

10. (canceled)

11. The phototherapy system of claim 10, wherein the processor circuitry is further configured to determine the light effect signal, such that the phototherapy parameters are modulated to ensure that a predetermined amount of the phototherapeutic light is being absorbed by the target tissue.

12. A spectral absorption measurement device for determining spectral absorption characteristics of tissue and for adjusting phototherapy parameters used by a light source for generating phototherapeutic light having at least one phototherapeutic wavelength based on the determined spectral absorption characteristics, the spectral absorption measurement device comprising:
detector assemblies, wherein each of the detector assemblies is keyed to a wavelength of light and includes:
a light emitter configured to emit identifying light including the keyed wavelength of light, such that the identifying light interacts with the tissue; and
at least one light sensor sensitive to the keyed wavelength of light and configured to be positioned relative to the tissue, such that returning light is detected by the at least one light sensor, wherein:
the returning light comprises a portion of the identifying light interacting with the tissue that is returned from the tissue; and
processor circuitry configured to:
determine an input property of the emitted identifying light, wherein the input property is an optical power of the emitted identifying light;
determine an output property of the detected returning light, wherein the output property is an optical power of the detected returning light;
determine as optical tissue properties an effect of the tissue on the keyed wavelengths of each of the detector assemblies, wherein the optical tissue properties is determined based on the difference between the input property and the output property;
based on the determined optical tissue properties, determine as the spectral absorption characteristics an effect of the tissue on the at least one phototherapeutic wavelength, wherein:
the spectral absorption characteristics are determined by the processor circuitry comparing the optical tissue properties to predetermined relationships between spectral absorption and tissue parameters to identify an effect of the tissue on the therapeutic light; and
the effect of the tissue includes a predicted ratio of effective light comprising the phototherapeutic light absorbed by the target tissue and ineffective light comprising the phototherapeutic light not absorbed by the target tissue; and
output a light effect signal based on the determined spectral absorption characteristics describing modifications to the phototherapy parameters.

13. The spectral absorption measurement device of claim 12, wherein each of the detector assemblies includes at least three light sensors and the at least three light sensors encircle the light emitter of the detector assembly.

14. The spectral absorption measurement device of claim 12, wherein at least one of the input property of the emitted identifying light or the output property of the detected returning light includes:
at least one of a wavelength, intensity, or power.

15. The spectral absorption measurement device of claim 12, wherein:
the detector assemblies are classified into one of at least two groups;
for each of the detector assemblies in a same group:
the identifying light emitted by the light emitters includes a same keyed wavelength of light; and
the light sensors are sensitive to the same keyed wavelength of light.

16. The spectral absorption measurement device of claim 15, wherein the detector assemblies of the at least two groups are spatially intermixed.

17. The spectral absorption measurement device of claim 12, wherein a combination of the keyed wavelengths of the detector assemblies includes:
at least one of the at least one phototherapeutic wavelength; and
at least one probing wavelength not included in the at least one phototherapeutic light and for determining the optical tissue properties.

18. The spectral absorption measurement device of claim 12, wherein each of the detector assemblies includes multiple keyed wavelengths of light, such that:
the at least one light sensor of each of the detector assemblies is sensitive to the multiple keyed wavelengths of the detector assembly; and
the identifying light of the light emitter of each of the detector assemblies includes the multiple keyed wavelengths of the detector assembly.

19. The spectral absorption measurement device of claim 12, wherein the processor circuitry is further configured to:
analyze across the detector assemblies the output property of the detected returning light to identify from the keyed wavelengths as a reduced wavelength of light the key wavelength that is more preferentially absorbed by the tissue; and
determine a physiological property of the tissue based the identified reduced wavelength of light.

20. The spectral absorption measurement device of claim 19, wherein the effect of the tissue includes a predicted ratio of effective light comprising the phototherapeutic light absorbed by the target tissue and ineffective light comprising the phototherapeutic light not absorbed by the target tissue.
